(19) United States
(12) Patent Application Publication
(10) Pub. No.: US 2008/0281415 A1
(43) Pub. Date: Nov. 13, 2008

(54) SECOND ELASTIC HINGE ACCOMMODATING INTRAOCULAR LENS

(75) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C&C VISION INTERNATIONAL LIMITED

(21) Appl. No.: 11/925,313

(22) Filed: Oct. 26, 2007

Related U.S. Application Data
(60) Provisional application No. 60/894,632, filed on Mar. 13, 2007.

(51) Int. Cl. A61F 2/16

(52) U.S. Cl. 623/6.23

(57) ABSTRACT

An accommodating intraocular lens where the optic is moveable relative to the ends of the extended haptic portions. The lens comprises an optic made from a flexible material combined with haptics capable of multiple flexions without breaking. The haptics having in longitudinal cross section wide and deep hinges adjacent the optic to better allow the elastic hinges to “stretch” when the optic is subjected to posterior pressure thus allowing the optic to move forward relative to both the outer and inner ends of the haptics. When this movement is combined with the movement of the optic relative to the outer ends of the haptics, the anterior movement of the whole lens, and a change in shape of the optic, the refractive power of the eye is further enhanced.

### CROSS-REFERENCE TO RELATED APPLICATION

This application claims priority from Ser. No. 60/894,632 filed Mar. 13, 2007, the disclosure of which is incorporated herein by reference.

### BACKGROUND

Intraocular lenses have for many years had a design of a single optic with loops attached to the optic to center the lens and fixate it in the empty capsular bag of the human eye. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a square edge into which were inserted loops to center the lens in the eye and fixate it within the capsular bag.

Recently accommodating intraocular lenses have been introduced to the market, which generally are modified plate haptic lenses. A plate haptic lens may be referred to as an intraocular lens having two or more plate haptics joined to the optic.

Flexible acrylic material has gained significant popularity among ophthalmic surgeons; however some acrylic materials are incapable of multiple flexions without fracturing. In 2003, more than 50% of the intraocular lenses implanted had acrylic optics. Flexible hydrogel and collamer lenses have also been introduced.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally a groove or hinge can be placed across the plate adjacent to the optic as part of the lens design to facilitate movement of the optic relative to the ends of the haptics.

### SUMMARY OF THE INVENTION

According to a preferred embodiment of this invention, an accommodating lens comprises a lens with a flexible solid optic attached to which are two or more extended portions. The extended portions, haptics, can be plates FIGS. 1, 2, 8 or loops FIGS. 4, 5, 6, 7 and 9, which can be open or closed, each capable of multiple flexions without breaking. The haptics preferably having fixation and centration features at their distal ends, e.g., protuberances on one or both sides front or back. The extended portions are designed such that upon constriction of the ciliary muscle with its associated increase in vitreous cavity pressure, the extended portions are configured to move peripherally or outward by sliding in the capsular bag pockets. This can be accompanied by making the proximal end of the haptics narrower than the distal end or by making the haptics with parallel sides. Such a lens design upon ciliary muscle contraction moves peripherally and anteriorly with an increase in vitreous cavity pressure. Hinges or grooves across the extended portions adjacent to the optic facilitate the movement of the optic relative to the ends of the extended portions. The wide base of the flexible hinge allows stretching of the elastic base of the hinge in the longitudinal axis of the lens with ciliary muscle contraction and an increase of vitreous cavity pressure, thus allowing anterior movement of the optic relative to both ends of the haptics in addition to the anterior movement of the posteriorly vaulted optic relative to the outer ends of the haptics by a flattening of the angle between the lens optic and haptics.

In the human, the whole crystalline lens moves forward upon ciliary muscle contraction, which also occurs with accommodating lenses. During ciliary muscle contraction the vitreous pressure increases and this can move the optic of a flexible lens, with an elastic hinge, forward relative to both ends of haptics.

In addition, with constriction of the ciliary muscle and relaxation of the zonules, the peripheral radial pull on the lens is reduced and the fibrosed capsular bag can then exert a central radial longitudinal force on the lens which can cause a change in shape of the optic such that, in addition to optic movement, it adds power to the change in the eye's refraction. This can occur by either deformation of the haptic or by an increase in the thickness of the optic center with a decrease in its radius of curvature.

The accommodating power of the accommodating IOL upon ciliary muscle contraction can therefore be the combination of four factors; namely:

a) The anterior movement of the whole lens since that occurs in the human crystalline lens.

b) An increase in vitreous cavity pressure that causes the posterior vaulted haptics in the eye to move peripherally, thereby allowing the posteriorly vaulted lens optic to change the angle between it and the haptic and to move forward relative to the outer ends of the haptics.

c) The anterior movement of the optic relative to both the outer and inner ends of the haptics by stretching of the elastic base of the hinge.

d) Deformation of the thin lens optic.

The various mechanisms can act alone or in combination and are mainly dependent on the design of the haptics. The haptics can be either a plate or loop design, and the loops either open or closed. The preferable design is a plate. The plates may have protrusions on the anterior or posterior or both surfaces.

In some embodiments, the haptic sides are parallel, thereby allowing them to slide along the capsular bag pockets upon constriction of the ciliary muscle and relaxation of the zonules. The vitreous pressure pushes on the IOL thereby flattening the posteriorly vaulted lens to move the optic forward relative to the outer ends of the haptics. The bag with its slack zonules is then deformed in the long axis of the lens.

When the distal ends of the plate haptics are wider than the proximal ends, this gives a wider area of contact of the capsular bag pocket with the plates and stabilizes the lens to give a more predictable distance vision. The narrow proximal end adjacent to the optic when it has a hinge, presents a less resistant hinge base. The hinge base, between the two walls of the hinge, preferably is widened to allow it to stretch like an elastic band. The shape of the plate allows easier movement peripherally of the plate since a narrow part of the plate is moving into a wider pocket. Since the haptic itself is flexible and elastic, it too can stretch to allow additional anterior movement of the optic. This can also occur without the need for a hinge.

Accordingly, features of the present invention are to provide an improved form of accommodating lens.

### BRIEF DESCRIPTION OF THE DRAWINGS

FIG. 1 is a front elevational view of a preferred embodiment of the present invention.

FIG. 2 is a side view.

FIG. 3 is a detail view of a hinge with a widened hinge base.

FIGS. 4-12 show variations of the haptics.

According to the present invention, the optic is of a foldable, flexible silicone, acrylic, collamer, or hydrogel material and the haptic plates are of a foldable material that will withstand multiple foldings and stretchings without damage, e.g., silicone, hydrogel or collamer. Preferably, the end of the plate haptics essentially have T-shaped fixation devices and may be hinged to the square edged optic.

### DESCRIPTION OF THE PREFERRED EMBODIMENT

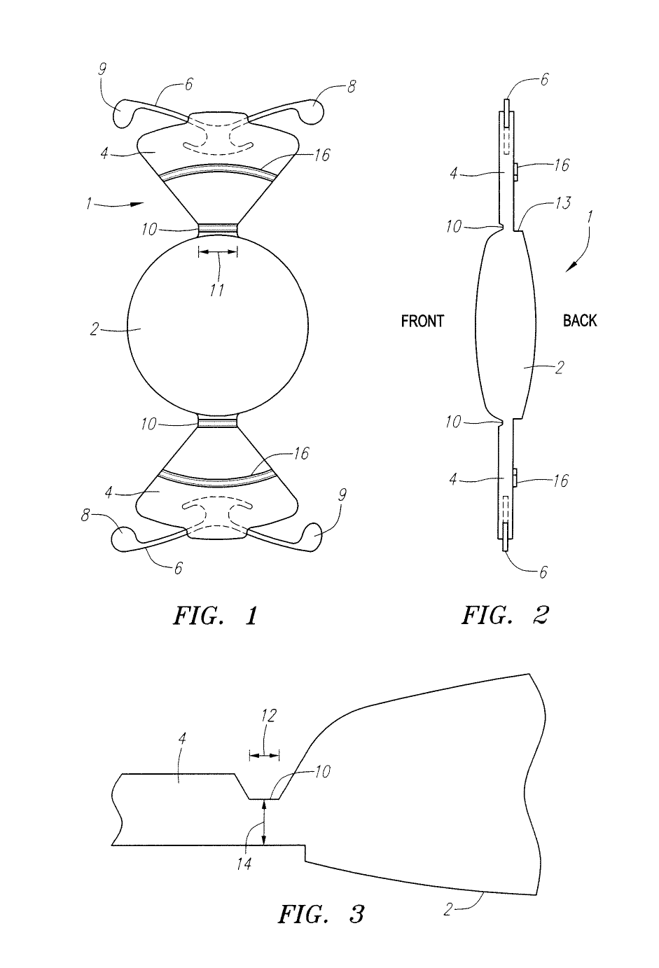

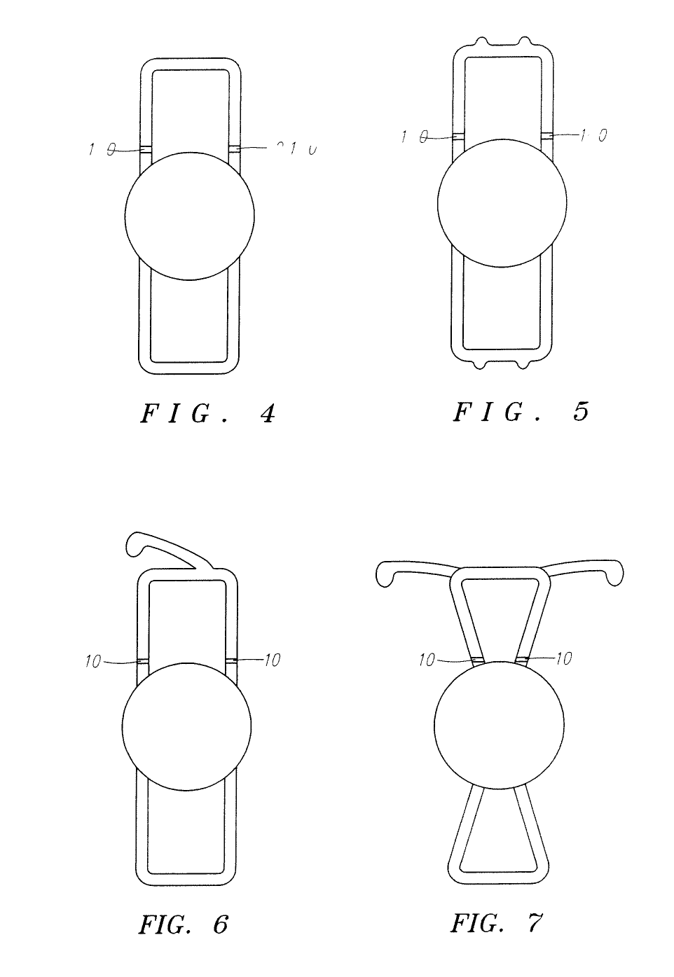

Turning now to FIG. 1, a preferred embodiment is illustrated in detail comprising an intraocular lens 1 formed as a flexible solid optic 2 preferably made of silicone, and flexible extending portions 4 of any suitable form but preferably silicone triangular plate haptics with the narrow bases adjacent to the optic which are capable of multiple flexations without damage. The optic 2 and haptics 4 preferably are uniplanar until implanted into the eye, and two or more haptics 4 extend distally from the optic 2. The haptics may be plates or loops which may be open or closed. Fixation and centration fingers as seen in FIG. 6, or loops or protuberances as seen in FIGS. 9-11 can be provided at the distal ends of the hinged haptics 4.

A typical length for the lens from loop tip to loop tip is 10.0-12.50 mm, and the optic 2 typically is a 4.5-7.0 mm diameter optic. The haptic plate length preferably is 10.0-11.5 mm. The fingers 6 preferably are approximately 5.0 mm wide and comprise four-point fixation loops that flex when the lens is placed into the capsular bag or put into any insertion cartridge. The two ends 8 of the four-point fixation loop have a slightly different configuration and aid in indicating to the surgeon that the lens is right side up with the hinges in a proper position.

Importantly, the haptics 4 have a triangular shape, narrower adjacent to the optic, and wider at the outer ends. Hinges 10 are provided between the haptics 4 and the outer periphery of the optic 2, and it is particularly desirable to have a wide elastic base 12 (FIG. 3) to the narrow hinge width 11 tangential to the optic to further allow the optic 2 to move forward more by stretching of the thin elastic hinge base with the increase in vitreous cavity pressure which allows more anterior movement of the optic than in current designs such as for example that shown in U.S. Pat. No. 6,398,126. A typical hinge width 11 is 1.0-5.0 mm, and preferably with a hinge base width longitudinally as indicated by arrow 12 of 0.06-0.4 mm, and preferably 0.12 mm, as seen in FIG. 3. The thickness 14 of the base 12 preferably is 0.5-1.5 mm. The wider hinge base 12 stretches like an elastic band to facilitate greater anterior movement of the optic 2.

The hinges 10 are on the anterior side of the implanted lens and the round end 8 of loops 6 on the right as seen in FIG. 1 indicates that the hinge is uppermost. End 8 is round whereas the end on the opposite loop is oval. The wider loops 6 and wide peripheral plates stabilize the lens and therefore provide better and more predictable distance vision.

Preferably the optic and plate haptics are silicone and the loops 6 are polyimide.

There can be a sharp 360-degree edge 13 around the posterior surface of the optic 2, to reduce the migration of cells across the posterior capsule of the lens postoperatively and thereby reduce the incidence of posterior capsular opacification and the necessity of YAG posterior capsulotomy. There may also be one or more ridges 16 as seen in FIGS. 1 and 2 across the plate to further prevent posterior capsular opacification. The optic may have one or both surfaces that are polyspheric, aspheric or Fresnell.

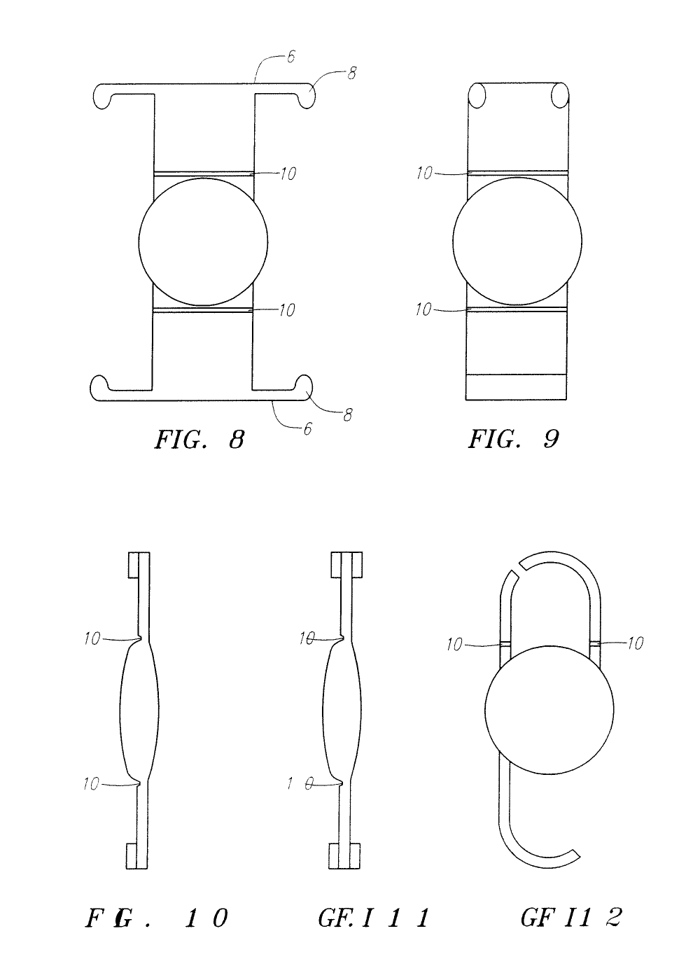

FIGS. 4-12 illustrate variations with different haptics and fixation devices. Hinges 10 as shown can be on one or both sides of a plate haptic or on the sides of the loop haptics.

As is well known in the art, the intraocular lens 1 such as that in the drawings is implanted in the capsular bag of the eye after removal of the natural lens. The lens is inserted into the capsular bag through a generally circular opening torn in the anterior capsular bag of the human lens after passing through a small opening in the cornea or sclera. The outer ends of the haptics 4, or loops 6, are positioned in the cul-de-sac of the capsular bag. The outer ends of the haptics, or the loops, are in close proximity with the bag cul-de-sac, and loops are deflected centrally to conform with the inner surface of the capsular bag. The ends or knobs of the loops are provided on the outer end portions of the loops 6 for fixation to secure the lens in the capsular bag or cul-de-sac with fibrosis, which develops in the capsular bag following the surgical removal of the central lens cortex and nucleus.

The inner ends of the loops 6 may be either integrally formed from the same material as the haptics 4 or the loops may be of a separate material such as polyimide. The loops, if formed of a separate material, are molded into the terminal portions of the haptics 4 or if the lens is lathe cut, attached after the lens body is fabricated.

Accordingly, there has been shown and described a lens that ideally comprises a silicone optic and silicone haptic plates with loops at their distal ends that can be of a different material than the plate, and provide fixation and centration of the lens in the eye. The haptics designed for movement along the tunnel formed by the fusion of the anterior and posterior capsules of the human capsular bag. The lens having wide elastic bases to the hinges such that they can stretch like a rubber band to allow the optic to move by flexion of the hinge and stretching of its wide elastic base.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

What is claimed is:

1. An accommodating intraocular lens comprising a flexible solid optic and attached flexible extended portions comprising narrow haptics, designed such that the optic can move backward and forward relative to the extended portions and whereby the narrow haptics adjacent to the optic can slide in pockets of the eye formed by the fusion of the anterior and posterior capsules and have a thinned hinge area with a widened elastic hinge base such that upon an increase in posterior pressure, the thinned and widened hinge elastic area can stretch like a rubber band to further aid anterior movement of the optic relative to both the outer and inner ends of the haptics.

2. A lens according to claim 1 wherein the haptics are relatively narrow adjacent the optic and are wider distally.

3. A lens according to claim 1 wherein the haptics have parallel sides.

4. A lens according to claim 3 wherein the haptics have a thinned area adjacent to the optic.

5. A lens according to claim 4 wherein the thinned area is a hinge.

6. A lens according to claim 4 wherein the thinned area is a hinge and is trough shaped and has a wide base connecting the two sides of a hinge.

7. A lens according to claim 4 wherein the thinned area is a shallow groove.

8. A lens according to claim 1 wherein one or more fixation/centration fingers are on the ends of the extended portions.

9. A lens according to claim 8 wherein the fixation/centration fingers indicate the correct side up of the lens in the eye.

10. A lens according to claim 8 wherein the fingers are designed to extend beyond the diameter of the capsular bag and are flexible to bend to conform to the bag diameter.

11. A lens according to claim 1 wherein the extended portions include loops and/or fixation devices of polyimide.

12. A lens according to claim 11 wherein the loops have a fixation element of a different shape on their proximal ends to enhance centration and fixation of the lens within the capsular bag.

13. A lens according to claim 1 wherein the lens is made of an optical material or a combination of optical materials that are inert, e.g. silicone, HEMA, acrylic, collamer, or other material.

14. A lens according to claim 1 wherein fixation loops or fingers are on the ends of the haptics and are made of a different material than the lens, e.g. polyimide, PMMA, Prolene, etc.

15. A lens according to claim 13 where the lens optic is made of a different material than the haptics.

16. A lens according to claim 1 wherein the optic has a 360-degree square edge on its posterior surface.

17. A lens according to claim 1 wherein the flexible optic is capable of a shape change that increases its refractive power with constriction of the ciliary muscle.

18. A lens according to claim 1 wherein the optic has one or both surfaces that are polyspheric.

19. A lens according to claim 1 wherein the optic has one or more surfaces that are aspheric.

20. A lens according to claim 1 wherein the optic diameter is from 4 to 7 mm.

21. A lens according to claim 1 wherein the optic moves relative to the outer ends of the haptics.

22. A lens according to claim 1 wherein the haptics are plates that have protuberances on either their anterior or posterior sides or both sides.

23. A lens according to claim 1 wherein the extended portions are opened or closed loops.
